(19) United States
(12) Patent Application Publication
(10) Pub. No.: US 2016/0263344 A1
(43) Pub. Date: Sep. 15, 2016

(54) VOLATILE FLUID DISPENSER WITH ROTATIONAL ACTIVATION

(71) Applicant: The Dial Corporation, Scottsdale, AZ (US)

(72) Inventors: Kevin Hafer, Chandler, AZ (US); James Clark, Chicago, IL (US); Matthew Freeborn, Scottsdale, AZ (US)

(21) Appl. No.: 14/644,363

(22) Filed: Mar. 11, 2015

(51) Int. Cl. A61M 21/02; A01M 1/20; A01M 13/00

(52) U.S. Cl. CPC A61M 21/02; A01M 13/00; A01M 1/20; A61M 2021/0016

(57) ABSTRACT

Methods and devices are provided for fluid dispensers with rotational activation mechanisms. The device may include a reservoir that is sealed to prevent an escape of fluid. The sealed reservoir includes an internal barrier. The device includes a wicking assembly that includes a wick and a rotational element. Rotation of the rotational element breaks the internal barrier of the sealed reservoir and allows the fluid in the reservoir to contact the wick.

### FIELD OF THE INVENTION

The present invention generally relates to dispensers for volatile material, and more particularly relates to apparatuses and methods for activation of dispensers for volatile liquids.

### BACKGROUND OF THE INVENTION

Aqueous and non-aqueous liquid air fresheners have gained popularity for providing a pleasant aroma to an environment. There are a variety of types of dispensers for aqueous and non-aqueous liquid air fresheners, which dispensers may also be used to assist in the evaporation of other volatile liquids. Such other volatile liquids may include aqueous scent mixtures, insect repellants, or odor-based deterrents for animals or humans.

Some dispensers for volatile fluids, such as aqueous and non-aqueous liquid air fresheners, use electricity to drive the evaporation of the volatile fluids. Conversely, other dispensers for volatile fluids do not use electricity, and may provide a large surface area from which the volatile fluid may evaporate.

During handling and storage, some non-electric dispensers may maintain the volatile fluid separate from the large evaporative surface area. During use, these dispensers may require multiple assembly steps to put the volatile fluid in contact with the large evaporative surface area. The multiple assembly steps may be cumbersome for a user and lead to a less than satisfactory user experience. In an alternate approach the volatile fluid may contact the large evaporative surface area. In this approach premature evaporation during handling and storage is prevented by using a cap or other cover to seal the assembly from the outside environment. However, this second approach is also not desirable due to aesthetic and leakage concerns.

Accordingly, it is desirable to provide a dispenser for volatile fluids that may be easily assembled by a user. In addition, it is desirable for such a dispenser to maintain the volatile fluid separate from the evaporative surface area until a user assembles the unit. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A device is provided for activating a volatile reservoir using a rotational force. The device comprises a container assembly and a wicking assembly. The container assembly comprises a reservoir that is sealed to prevent an escape of a volatile fluid, and an internal barrier. The wicking assembly comprises a wick and a rotational element. The rotation of the rotational element breaks the internal barrier of the container assembly, which allows the volatile fluid in the reservoir to contact the wick of the wicking assembly.

A method is provided for producing an apparatus to activate a reservoir. The method comprises providing a container assembly comprising a reservoir, placing a fluid into the reservoir, applying a seal to contain the fluid within the reservoir, and affixing a wicking assembly to the container assembly, where the wicking assembly comprises a wick and a rotational element. The rotation of the rotational element breaks an internal barrier separating the fluid in the reservoir from the wick of the wicking assembly.

A consumer product is provided for dispensing a volatile fluid. The consumer product comprises a container assembly, a wicking assembly, and an outer housing assembly. The container assembly comprises a reservoir that is sealed to contain a volatile fluid, where the sealed reservoir comprises an internal barrier. The wicking assembly comprises a wick, a rotational element and a fitment. The outer housing assembly comprises a rib to engage the rotational element and a slot into which the container assembly and the wicking assembly may be inserted. The container assembly, wicking assembly and outer housing assembly are provided such that the engagement of the rib of the outer housing assembly with the rotational element of the wicking assembly couples the rotation of the rotational element to the outer housing assembly, the rotation of the rotational element relative to the container assembly breaks the internal barrier of the container assembly, and the breakage of the internal barrier of the container assembly allows the volatile fluid contained in the reservoir to contact the wick of the wicking assembly.

### BRIEF DESCRIPTION OF THE DRAWINGS

The present invention will hereinafter be described in conjunction with the following drawing figures, wherein like numerals denote like elements, and

FIG. 1 is a cross-sectional side-view diagram of a container assembly and a wicking assembly prior to activation, according to an example of the principles described herein;

FIG. 2 is a cross-sectional side-view diagram of the container and wicking assemblies of FIG. 1 after activation, according to an example of the principles described herein;

FIG. 3 is a cross-sectional front-view diagram of a container assembly and a wicking assembly prior to activation, according to an example of the principles described herein;

FIG. 4 is a cross-sectional front-view diagram of the container and wicking assemblies of FIG. 3 after activation, according to an example of the principles described herein;

FIG. 5 is a cross-sectional side-view diagram of the container and wicking assemblies of FIG. 3 after activation, according to an example of the principles described herein;

FIGS. 6A-6D are cross-sectional side-view and top-view diagrams of a wicking assembly prior to activation, according to an example of the principles described herein;

FIG. 7 is a cross-sectional side-view and top-view diagram of the wicking assembly of FIGS. 6A-6D after activation, according to an example of the principles described herein;

FIG. 8 is a cross-sectional bottom-view diagram of a container assembly and a wicking assembly inserted into an outer housing assembly prior to activation, according to an example of the principles described herein;

FIG. 9 is a cross-sectional bottom-view diagram of the container and wicking assemblies of FIG. 8 inserted into the outer housing assembly of FIG. 8 after activation, according to an example of the principles described herein;

FIG. 10 is a cross-sectional side-view diagram showing the partial insertion of a container assembly and a wicking assembly into an outer housing assembly, according to an example of the principles described herein;

FIG. 11 is a chart showing the angle of a reservoir of a container assembly and a rotational element as a function of insertion depth relative to an outer housing assembly, according to an example of the principles described herein;

FIG. 12 is a chart showing the angle of a reservoir of a container assembly and a rotational element as a function of insertion depth relative to an outer housing assembly, according to an example of the principles described herein;

FIG. 13 is a chart showing the angle of a reservoir of a container assembly and a rotational element as a function of insertion depth relative to an outer housing assembly, according to an example of the principles described herein; and

FIG. 14 is a chart showing the angle of a reservoir of a container assembly and a rotational element as a function of insertion depth relative to an outer housing assembly, according to an example of the principles described herein; and

FIG. 15 is a flowchart of a method of making a volatile fluid dispenser, according to an example of the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As noted above, volatile fluid dispensers may be a popular way to imbue an environment with a pleasant aroma by facilitating evaporation of the volatile fluid. Volatile fluid dispensers may promote the evaporation of the volatile fluid by either electric or non-electric means.

Volatile fluids may provide an olfactory sensation. For example, the volatile fluid may include a fragrance, such that the dispenser dispensing the volatile fluid may cause the environment around the dispenser to become fragrant with the scent of the fragrance. In another example, the volatile fluid may include an insect repellent, such that the dispenser dispensing the volatile fluid may cause insects to avoid the environment around the dispenser. In this example, the volatile fluid dispenser may not produce an olfactory sensation in humans. In yet another example, the volatile fluid may have an unpleasant odor, and the volatile fluid dispenser may thus cause the surrounding environment to have an unpleasant odor, whereby acting as a deterrent for humans or animals. A volatile fluid dispenser that produces an unpleasant odor may be used, for example, to deter house pets from furniture or areas of a house where they may be unwanted or unsafe. A volatile fluid dispenser that is intended to act as a deterrent to non-human creatures (for example, pets or insects) may contain a volatile fluid with an odor that is imperceptible to humans, or that may be perceived as pleasant by humans. In a further example, a volatile fluid may also include aromatherapeutic agents. In a still further example, a volatile fluid may also include mood-enhancing substances. It is also possible for a volatile fluid to contain more than one of the aforementioned properties or additives; for example, a volatile fluid could include an aromatherapeutic agent, a fragrance and an insect repellent. In another example, a volatile fluid could contain both an aromatherapeutic agent and a mood-enhancing substance. The volatile fluid may also contain a carrier fluid, which may be an oil, water, an organic solvent, a silicone, or combinations thereof.

The ease of assembly of a volatile fluid dispenser may be a factor in consumer appeal. For example, aromatic volatile fluids may cause skin irritation and therefore it may be desirable to prevent contact of the volatile fluid with skin. Accordingly, it is desirable to provide a volatile fluid dispenser that is easy to assemble and whose assembly does not risk spilling a volatile fluid contained therein.

The present specification is directed to activation methods and mechanisms for volatile fluid dispensers, and may be used in either electric or non-electric volatile fluid dispensers. The present specification provides a novel means to maintain the volatile fluid separately from the elements that promote the evaporation of the volatile fluid, while also providing facile activation by a user using a rotational force.

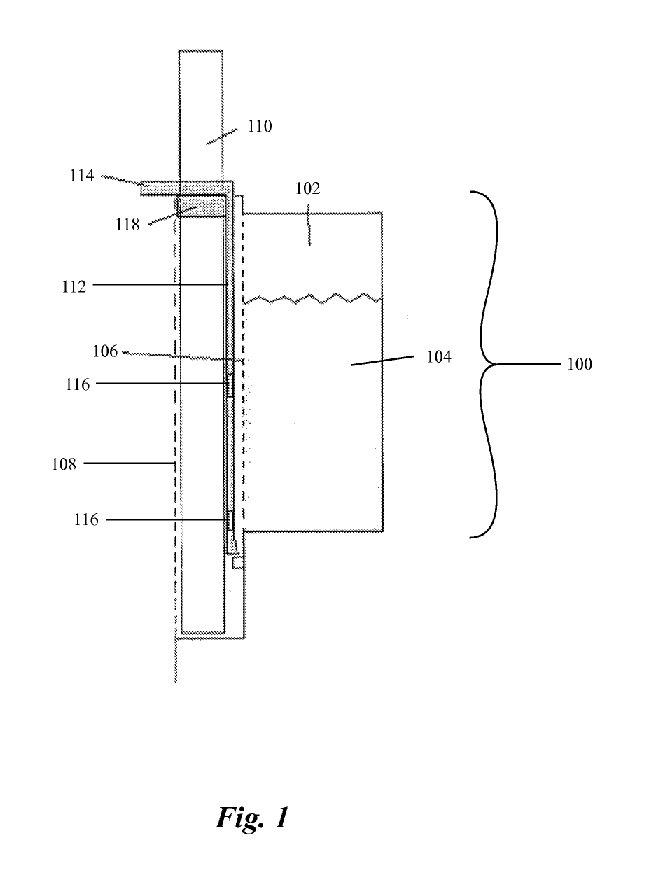

Turning now to the figures, FIG. 1 is a cross-sectional side-view diagram of a container assembly and a wicking assembly (collectively, 100) according to an example of the principles described herein, shown prior to activation. The container assembly of FIG. 1 includes a reservoir (102), which contains a volatile fluid (104). The container assembly of FIG. 1 also includes an internal barrier (106), which may be broken upon activation of the volatile fluid dispenser. The container assembly of FIG. 1 also includes a second barrier (108). The wicking assembly of FIG. 1 includes a wick (110), and a rotational element (112). The rotational element (112) of FIG. 1 includes an activation lever (114) and two activation tabs (116). The container and wicking assemblies (100) of FIG. 1 additionally include a fitment (118), which may guide the rotation of the rotational element (112). The fitment (118) of FIG. 1 may be configured either to allow free rotation of the rotational element (112) or to limit the rotation of the rotational element (112) to a defined angle, such as 90 degrees)(°.

In the example of FIG. 1, the volatile fluid dispenser (100) may be activated by rotation of the rotational element (112) relative to the container assembly.

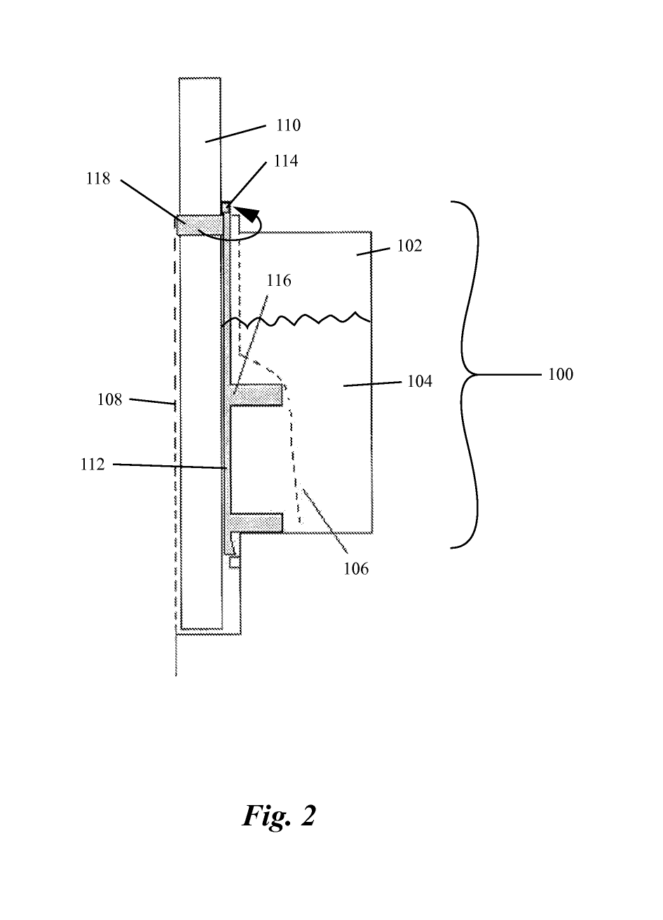

FIG. 2 is a cross-sectional side-view diagram of the container and wicking assemblies (100) of FIG. 1 after activation, according to an example of the principles described herein. As described above in FIG. 1, in the example of FIG. 2, the rotational element (112) includes two activation tabs (116). When the rotational element (112) is rotated relative to the container assembly (as indicated by the arrow), the activation tabs (116) may rupture the internal barrier (106) that separates the wick (110) from the volatile fluid (104) contained in the reservoir (102). In the example shown in FIG. 2, the internal barrier (106) which separates the wick (110) from the volatile fluid (104) contained in the reservoir (102) is flexible. However, in some examples, the internal barrier (106) may be a rigid material. A second barrier (108), which prevents leakage of the volatile fluid (104) contained within the reservoir (102) following activation of the volatile fluid dispenser (100), remains intact following activation, and may be either flexible or rigid. In some examples, the wick (110) of the wicking assembly has a lower floor than the reservoir (102); this height difference may allow the volatile fluid (104) to flow more quickly from the punctured reservoir (102), and allows the wick (110) to more effectively capture all of the volatile fluid (104) during the life of the container and wicking assemblies (100).

The example of FIGS. 1 and 2 is exemplary, and does not represent all types of volatile fluid dispensers (100) that may be used to dispense a volatile fluid according to the principles described herein.

The container assembly may be the portion that includes the reservoir (102). The reservoir (102) contains a volatile fluid (104), and may be sealed to prevent the premature escape of the volatile fluid (104). The seal of the reservoir (102) may be a surface that prevents the volatile fluid (104) contained in the reservoir (102) from prematurely escaping.

The container assembly may be provided as a single unit with a wicking assembly (100), such as that shown in FIG. 1. The wicking assembly may include a wick (110), which may draw in the volatile fluid (104) through capillary action to an emanator pad. An emanator pad may be an element with increased surface area to promote the evaporation of the volatile fluid (104) from its surface. In one example, the wick (110) is connected to, and considered to be a single unit with, the emanator pad. In another example, the wick (110) may engage an emanator pad that may be part of the container assembly or the outer housing assembly. In yet another example, the wick (110) also provides the emanator pad, a portion of the wick (110) that directly contacts the volatile fluid (104) may be considered the wick, while a portion of the wick (110) which promotes evaporation of the volatile fluid (104) may be considered the emanator pad.

The wick (110) and emanator pad may be composed of the same material, or may be composed of different materials. The wick (110) and emanator pad may be made of any material that is suitable for the distribution of a volatile fluid (104) into the surrounding environment. For example, the wick (110) and/or emanator pad may be a porous material that is either synthetic or naturally produced. In one example, the wick (110) and/or emanator pad is made of bamboo. In another example, the wick (110) and/or emanator pad is made from cotton. In yet another example, the wick (110) is synthetically produced, for example from a porous plastic. In yet another example, the wick (110) may be made of bamboo, while the emanator pad may be made from cotton. In another example, the wick (110) and/or emanator pad may be made from fiber. In a further example, the wick (110) and/or emanator pad may be made from a ceramic material of either natural or synthetic origin. In a still further example, the wick (110) and/or emanator pad may be made from wood. In another example, the wick (110) and/or emanator pad may be made from cellulose. In a further example, the wick (110) and/or emanator pad may be made from paper. It is also possible for the wick (110) and/or emanator pad to be made from combinations of suitable materials, either as layers or blends.

If the volatile fluid dispenser (100) uses electrical resistance to increase the rate of evaporation of the volatile fluid (104), the wick (110) and/or emanator pad may also include an electrically resistive material, such as a ceramic or metallic material, which may be either porous or non-porous. In another example, the volatile fluid dispenser (100) promotes an enhanced evaporation of the volatile fluid (104) by promoting faster exchange of air surrounding the emanator pad (which may be accomplished by an electrical device, and does not require any electrical contact with either the wick or the emanator pad).

Conversely, if the volatile fluid dispenser (100) does not use electricity, the rate of evaporation of the volatile fluid (104) into the surrounding environment may be controlled by the accessible surface area of the wick (110) and emanator pad, for example. In another example, the volatile fluid dispenser (100) does not use electricity and the rate of evaporation of the volatile fluid (104) into the surrounding environment is controlled by the size and number of ventilation openings in the outer housing assembly, thus making the rate of exchange of air around the wick (110) and/or emanator pad the controlling factor in the rate of evaporation of the volatile fluid (104).

The wicking assembly may include a rotational element (112). The rotational element (112) of the wicking assembly may be able to rotate in response to a rotational force. The rotational element (112) may either rotate with the wick (110), or may rotate independently of the wick (110). In some examples, the rotational element (112) may be disposed around a circular wick (110), while in other examples, the rotational element (112) may be able to rotate about an axis that is adjacent to the wick (110). The rotational element (112) may include an activation lever (114), and may also include an activation tab (116). In the example shown in FIG. 1, the rotational element (112) contains a single activation lever (114) and two activation tabs (116), which may be arranged such that a view along the axis of rotation has the activation tabs (116) aligned at an angle which is separated from the activation lever (114) by approximately 90°. In another example, the rotational element (112) contains two activation levers (114) disposed on opposite sides of the axis of rotation of the rotational element (112), and has two activation tabs (116) which are also disposed on opposite sides of the axis of rotation. In a further example, the rotational element (112) includes a single activation lever (114) and a single activation tab (116). While specific reference is made to specific numbers of activation levers (114) and activation tabs (116), the rotational element (112) may include any number of activation levers (114) and activation tabs (116).

For the purposes of the present specification, the term “activation lever” may be used broadly to encompass any structure capable of applying a rotational force to the rotational element (112), and may or may not be a lever. Additionally, the activation lever (114) may be engaged by the outer housing assembly, or may be rotated directly by the application of a rotational force by the user. For example, the activation lever (114) may be provided by a number of tabs that are disposed above the reservoir (102) of the container assembly. In another example, the activation lever (114) may be provided by a number of teeth, which may be placed either on the top or bottom of the reservoir (102) of the container assembly, and may engage the outer housing assembly by a corresponding set of teeth on the outer housing assembly.

The activation tabs (116) may be any type of protrusion from the axis of rotation of the rotational element (112), and may be a portion of the rotational element (112) that interacts with the container assembly upon rotation. The interaction of the activation tabs (116) with the internal barrier (106) of the container assembly may cause the internal barrier (106) of the container assembly to rupture, puncture or break, which may activate the volatile fluid dispenser (100) by allowing the volatile fluid (104) contained in the reservoir (102) to access the wick (110) of the wicking assembly. In one example, the activation tabs (116) are provided by rectangular protrusions from the rotational element (112). In another example, the activation tabs (116) are provided by an ovular flange; in this example, the rotation of the ovular flange causes the asymmetrical portions of the oval to change orientation, which may cause these asymmetrical portions to interact with the internal barrier (106).

The internal barrier (106) may be a surface that separates the wick (110) of the wicking assembly from the reservoir (102) containing a volatile fluid (104). This surface may prevent premature contact of the volatile fluid (104) with the wick (110) during handling and storage. The activation of the volatile fluid dispenser (100) may occur by breakage of the internal barrier (106). The breaking of the internal barrier (106) separating the volatile fluid (104) in the reservoir (102) from the wick (110) may occur by any means that destroys the integrity of the internal barrier (106), such that the volatile fluid (104) is allowed to contact the wick (110). For example, the internal barrier (106) may be shattered, ruptured, punctured, or otherwise manipulated to break the internal barrier (106) and allow the volatile fluid (104) to access the wick (110).

In one example, the seal applied to the reservoir (102) to contain the volatile fluid (104) and the internal barrier (106) that is broken to activate the volatile fluid dispenser (100) are the same surface. In another example, the seal applied to the reservoir (102) to contain the volatile fluid (104) and the internal barrier (106) that is broken to activate the volatile fluid dispenser (100) are different surfaces. In other words, the seal of the reservoir (102) and the internal barrier (106) may both be components of a container assembly which includes a reservoir (102), and each may fulfill a different function; depending on the arrangement of the elements of the volatile fluid dispenser (100), both the seal of the reservoir (102) and the internal barrier (106) may be provided by a single structure, or may be provided by different structures. In the example shown in FIG. 1, the internal barrier (106) may also be the seal that is applied to the reservoir (102).

The internal barrier (106) separating the wick (110) from a volatile fluid (104) contained within a reservoir (102) may be either flexible or rigid. Depending on the materials chosen for the internal barrier (106), either a flexible or rigid barrier may be more easily broken to activate the dispenser (l 00) than the other. A flexible internal barrier (106) may provide for more facile manufacturing of the container assembly, while a rigid internal barrier (106) may provide a more discrete break during activation.

The container assembly may also include a second barrier (108) that prevents leakage of the volatile fluid (104) from the reservoir (102). In the example shown in FIGS. 1 and 2, the second barrier (108) prevents leakage of the volatile fluid (104) from the reservoir (102) after the activation of the volatile fluid dispenser (100). In another example, the second barrier (108) prevents leakage of the volatile fluid (104) from the reservoir (102) during storage and transport. Thus, in some examples the second barrier (108) may have a direct interface with the volatile fluid (104) contained in the reservoir (102), while in other examples the second barrier (108) only has a direct interface with the volatile fluid (104) contained in the reservoir (102) following activation of the volatile fluid dispenser (100).

The second barrier (108) separating the volatile fluid (104) from the environment outside the volatile fluid dispenser (100) may also be either flexible or rigid, although the second barrier (108) may be configured so as to not be readily broken in order to prevent undesirable leakage of the volatile fluid (104). This second barrier (108) may be used to seal the reservoir (102) during manufacturing, or may similarly be used to provide a seal following the breakage of the internal barrier (106) during activation. This second barrier (108) may not occlude access between a wick (110) and a volatile fluid (104) contained within a reservoir (102). This second barrier (108) also may not occlude access between a wick (110) and the outside environment. Rather, this second barrier (108) may be used in order to ensure that the volatile fluid (104) flows from the reservoir (102) to the outside environment through the wick (110).

The container and wicking assemblies (100) may also include a fitment (118). A fitment (118) may be a component that may guide the rotation of the rotational element (112). In some examples, the fitment (118) may be part of the wicking assembly, while in other examples the fitment (118) may be part of the container assembly. In some examples, the fitment (118) may also include the internal barrier (106) that is broken during activation of the volatile fluid dispenser (100), while in the example of FIGS. 1-2, the fitment (118) is independent of the internal barrier (106). In one example, the fitment (118) is constructed so as to allow the rotational element (112) to rotate through an angle of 90°, and does not allow further rotation in order to protect the integrity of the second barrier (108). In another example, the fitment (118) allows the rotational element (112) to freely rotate, and guides just the axis of rotation of the rotational element.

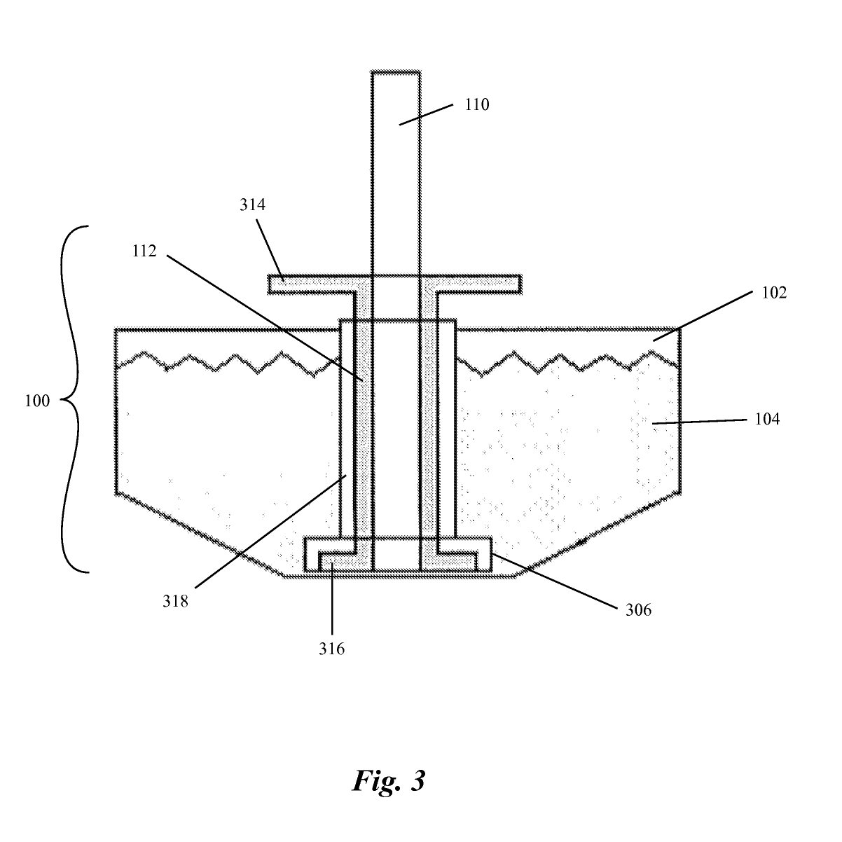

FIG. 3 is a cross-sectional front-view diagram of a container assembly and a wicking assembly prior to activation, according to an example of the principles described herein. The container assembly of this example includes a reservoir (102), which is filled with a volatile fluid (104). The container assembly of FIG. 3 includes a fitment (318), which may guide the rotation of the rotational element (112) to break the internal barrier (306). In this example, the fitment (318) may also provide a portion of the walls of the reservoir (102). The wicking assembly of this example includes a wick (110) and a rotational element (112). The rotational element (112) includes activation levers (314) and activation tabs (316). In the example of FIG. 3, the activation levers (314) may be shown as two opposing levers on opposite sides of a wick (110). In some examples, the activation tabs (316) may be in the form of an oval. In the example shown in FIG. 3, the internal surface (306) is different from the seal that is applied to the reservoir (102) to contain the volatile fluid during handling and storage.

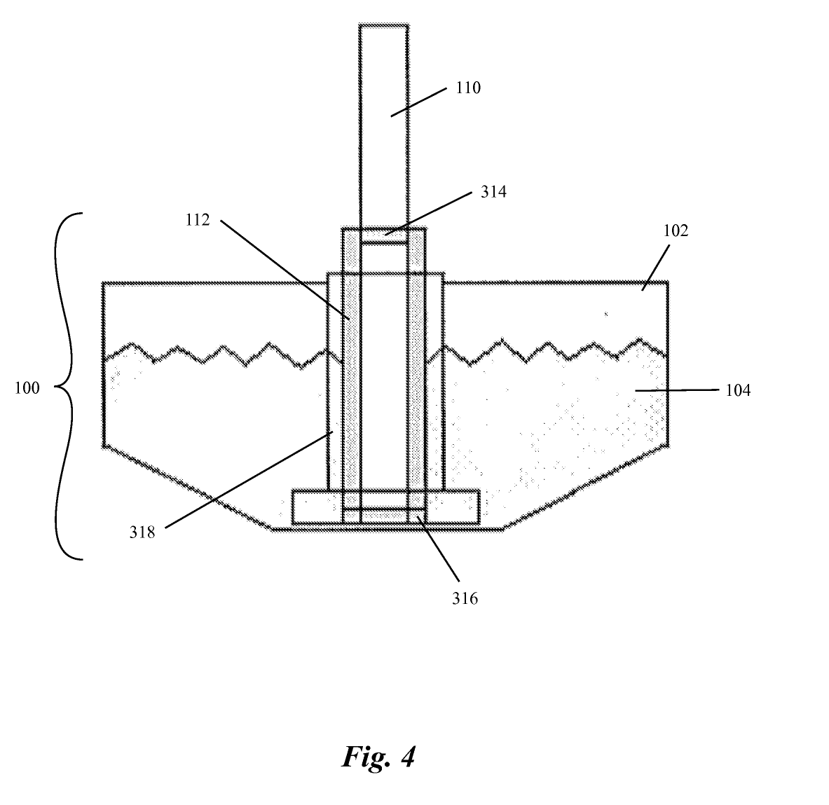

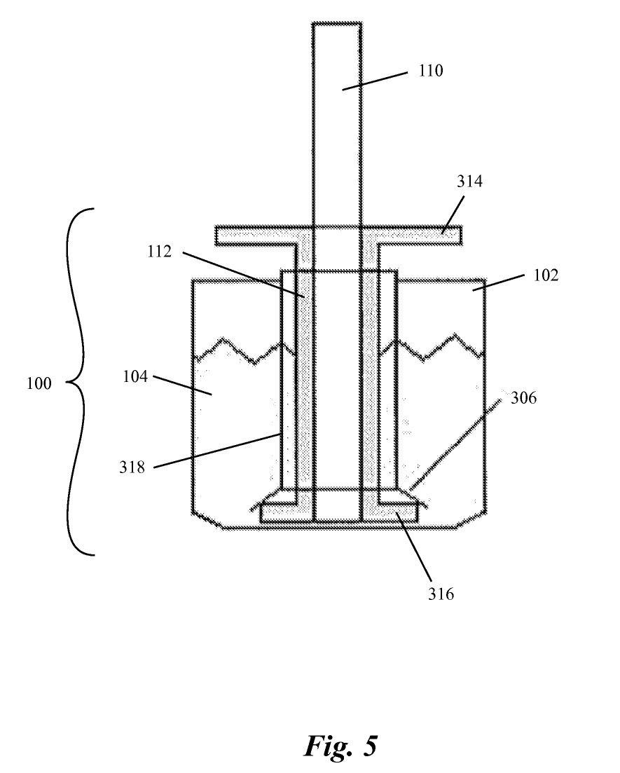

FIGS. 4 and 5 are cross-sectional diagrams of the container and wicking assemblies (100) of FIG. 3 after activation, according to examples of the principles described herein. For example, FIG. 4 shows the same cross-sectional front-view as FIG. 3, but the rotation of the rotational element (112) aligns the activation levers (314), as well as the activation tabs (316), with an axis perpendicular to the plane of the page. FIG. 5 shows a side-view of a cross-section of the container and wicking assemblies (100) of FIG. 3, and highlights the broken internal barrier (306) that allows the volatile fluid (104) contained in the reservoir (102) to contact the wick (110). The internal barrier (306) may be broken by the two activation tabs (316) that are designed for this purpose.

The example of FIGS. 3-5 is exemplary, and does not represent all types of volatile fluid dispensers (100) that may be used to dispense a volatile fluid (104) according to the principles described herein. For example, a volatile fluid dispenser (100) could be assembled to be similar to that shown in FIGS. 3-5, with the outer housing assembly residing under the container and wicking assemblies (100) and attached to the rotational element (112), such that a user may activate the volatile fluid dispenser (100) by rotating the outer housing assembly like a key (causing a corresponding rotation of the rotational element (112) and the activation tabs (316) which rupture the internal barrier (306) and activate the volatile fluid dispenser (100)). In another example, the outer housing may be omitted, and the user may activate the volatile fluid dispenser (100) of FIGS. 3-5 by rotating the activation levers (314) on either side of the wick (110), causing the rotation of the entire rotational element (112), including the activation tabs (316) that activate the dispenser.

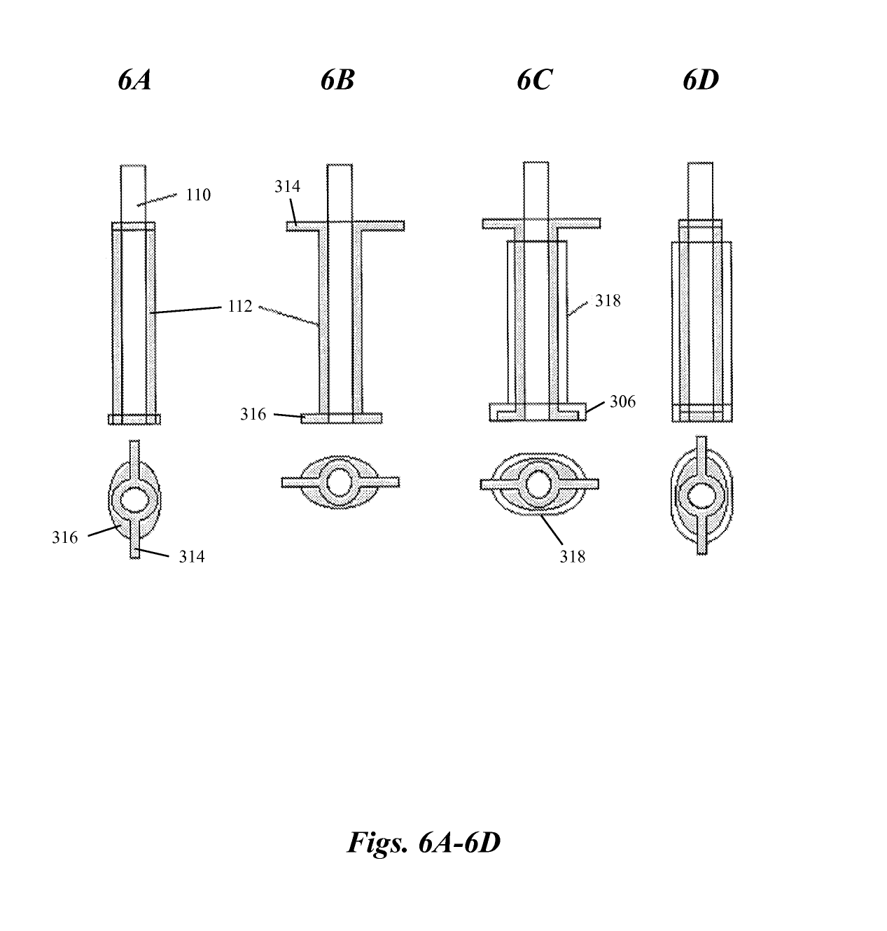

FIGS. 6A-6D are cross-sectional side-view and top-view diagrams of a wicking assembly prior to activation, according to an example of the principles described herein. FIG. 6A shows a side-view and top-view diagram of a wicking assembly. FIG. 6B shows a side-view and top-view diagram of a wicking assembly, in which the rotational element (112) is rotated 90° from the position shown in FIG. 6A. FIG. 6C shows a side-view and top-view diagram of a wicking assembly, in which the rotational element (112) is in the same position as in FIG. 6B. FIG. 6D shows a side-view and top-view diagram of a wicking assembly, in which the rotational element (112) is in the same position as in FIG. 6A. FIGS. 6C and 6D also show a fitment (318), which may be considered either part of the container assembly or the wicking assembly. The wick (110) and rotational element (112) may be similar to those shown in FIGS. 3-5. Underneath each side-view in FIGS. 6A-6D is a corresponding top-view showing the same position of the rotational element (112). FIGS. 6A-6D show the activation levers (314) protruding from the rotational element (112), as well as the ovular-shaped activation tabs (316) of the rotational element (112) which may break an internal barrier (306), which may be connected to the fitment (318), allowing access of the volatile fluid (FIG. 3, 104) contained within the reservoir (FIG. 3, 102) to the wick (110). While FIGS. 6A-6D indicate ovular-shaped activation tabs (316), any shape activation tab (316) may be used in the volatile fluid dispenser (FIG. 3, 100).

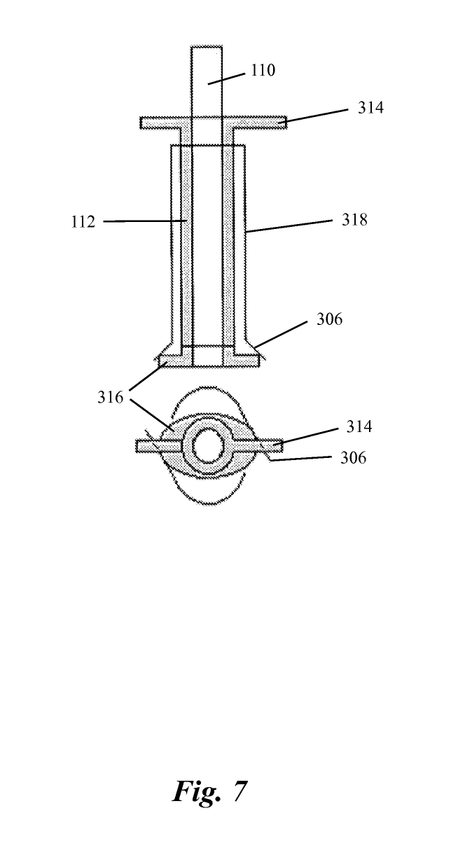

FIG. 7 is a cross-sectional side-view and top-view diagram of the wicking assembly of FIGS. 6A-6D after activation, according to an example of the principles described herein. Upon rotation of the rotational element (112), the activation tabs (316) may break an internal barrier (306). The breakage of the internal barrier (306) allows the volatile fluid (FIG. 3, 104) contained in the reservoir (FIG. 3, 102) to contact the wick (110). FIG. 7 shows two different ways that the activation tabs (316) can break the internal barrier (306). In the cross-sectional side-view, the bottom portion of the fitment (318) may be the thinnest—and weakest—portion. As a result, when the rotational element (112) is rotated by exerting a force on the activation levers (314), the activation tabs (316) break a portion of the internal barrier (306), causing this portion to move outward, as shown in the upper portion of the figure. In the top-view diagram, the thinnest portion of the fitment (318) may be along an axis parallel to the central axis, causing a break that is parallel to the axis of rotation of the rotational element (112). The bending interface shown at the edge of the internal barrier (306) in both the side-view and top-view diagrams may be provided by a living hinge, or another similar structure. While FIG. 7 may show two discrete types of break, any structure that allows the fluid contained within the reservoir to contact the wick (110) upon rotation of the rotational element (112) may be suitable. For example, it is also possible to construct the internal barrier (306) from a brittle material, such that when the internal surface (306) is broken, the internal surface (306) is no longer connected to the fitment (318).

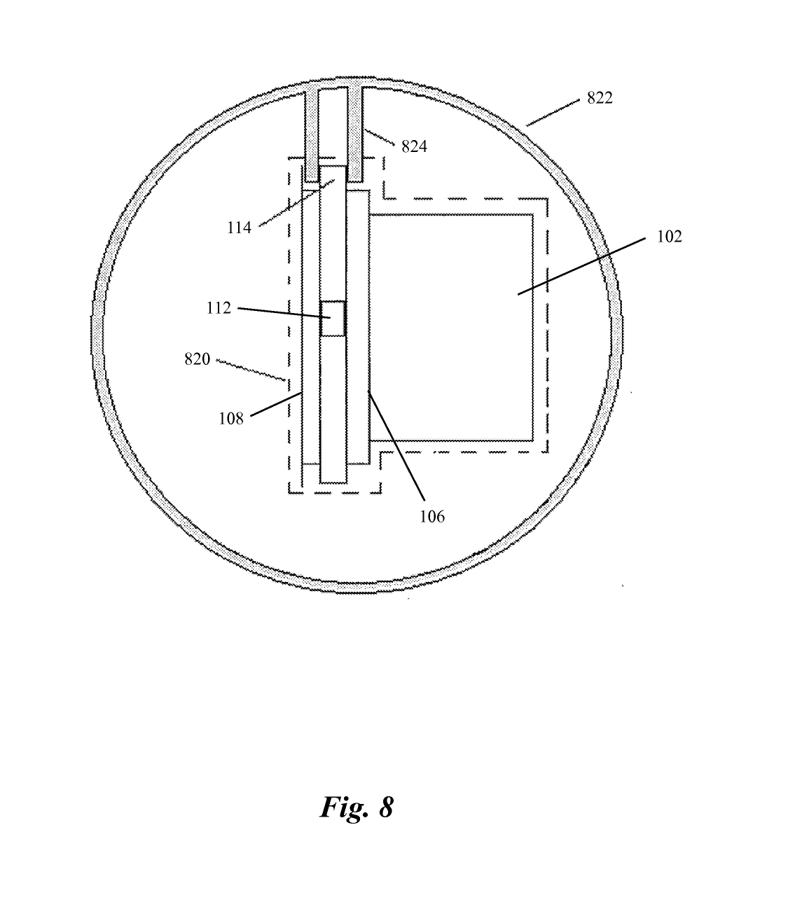

FIG. 8 is a cross-sectional bottom-view diagram of a container assembly and a wicking assembly (collectively, FIG. 1, 100) inserted into an outer housing assembly (822) prior to activation, according to an example of the principles described herein. FIG. 8 shows an outer housing assembly (822), including a slot (820) into which the container and wicking assemblies (FIG. 1, 100) are inserted. The outer housing assembly (822) may include a rib (824) to engage the activation lever (114) of the rotational element (112) of the wicking assembly. In one example, the rib (824) is linear along an axis perpendicular to the page. In another example, the rib (824) may be disposed about the interior of the outer housing assembly (822) such that the insertion of the container and wicking assemblies (FIG. 1, 100) into the outer housing assembly causes the rotational element (112) to rotate relative to the container assembly. For example, the rib (824) may be helical. This rotation activates the volatile fluid dispenser (FIG. 1, 100), breaking the internal barrier (106), while leaving the second barrier (108) intact, and allowing the volatile fluid within the reservoir (102) to access the wick (FIG. 1, 110) of the wicking assembly.

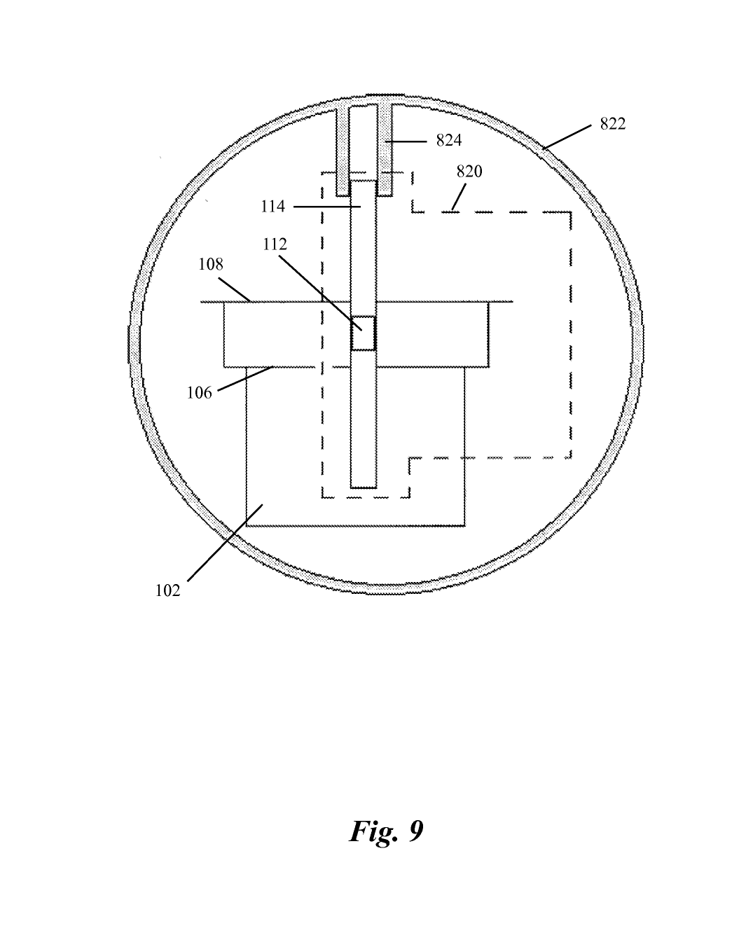

FIG. 9 is a cross-sectional bottom-view diagram of the container and wicking assemblies (100) of FIG. 8 inserted into the outer housing assembly (822) of FIG. 8 after activation, according to an example of the principles described herein. In FIG. 9, the container and wicking assemblies (FIG. 1, 100) are fully inserted into the outer housing assembly (822), and the container assembly is then rotated 90°. This 90° rotation of the container assembly within the outer housing assembly (822) causes the container assembly to rotate relative to the rotational element (112) as the rib (824) engages the activation lever (114) of the rotational element (112) of the wicking assembly, causing the rotational element (112) to remain stationary while the container assembly is rotated.

As with the previous figures, the example of FIGS. 8-9 is exemplary in nature, and does not represent all types, configurations, or shapes of outer housing assemblies (822) or wicking and container assemblies (FIG. 1, 100). For example, the slot (820) of the outer housing assembly (822) into which the container and wicking assemblies (FIG. 1, 100) may be inserted may be circular with a number of guiding slats disposed about the circular opening (820) of the outer housing assembly (822). One of the guiding slats may be for an activation lever (114) to engage a rib (824) of the outer housing assembly (822), and the others (if others are present) may guide the insertion of the container assembly into the outer housing assembly (822). If a number of guiding slats to guide the insertion of the container assembly into the outer housing assembly (822) are provided, these slats may also provide a locking means which may allow the outer housing assembly (822) to reversibly engage the container and wicking assemblies (FIG. 1, 100) using ribs similar to the rib (824) used to engage the activation lever (114).

The outer housing assembly (822) may provide some portion of the external surface of the assembled volatile fluid dispenser (FIG. 1, 100). In one example, the outer housing assembly (822) provides an external surface around the reservoir (102) and wick/emanator pad (FIG. 1, 110) of the container and wicking assemblies (FIG. 1, 100), which external surface may have slits or ventilation openings to allow the evaporation of the volatile fluid (FIG. 1, 104) into the surrounding environment. In another example, the outer housing assembly (822) provides an external surface around an emanator pad, but encloses a portion of the reservoir (102) that contains the volatile fluid (FIG. 1, 104). In yet another example, the outer housing assembly (822) is provided already attached to the container assembly, and the outer housing assembly (822) includes a tab on the base of the container assembly that allows a user to directly rotate the rotating element (112) of the wicking assembly. In this example, the outer housing (822) may not enclose either the reservoir or the wick/emanator pad (FIG. 1, 110) of the container and wicking assemblies (FIG. 1, 100).

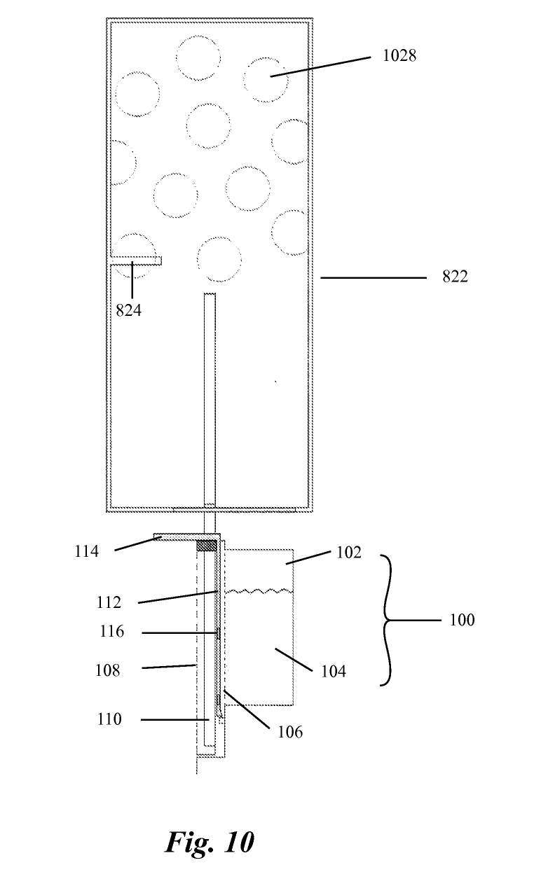

FIG. 10 is a cross-sectional side-view diagram showing the partial insertion of a container assembly and a wicking assembly (collectively, 100) into an outer housing assembly (822), according to an example of the principles described herein. The volatile fluid dispenser (100) of FIG. 10 may correspond to the container and wicking assemblies (100) of FIG. 1. The outer housing assembly (822) of FIG. 10 may correspond to the outer housing assembly of FIGS. 8-9. In the example shown in FIG. 10, the container and wicking assemblies (100) may be inserted entirely within the outer housing assembly (822) and then rotated 90° in order to activate the volatile fluid dispenser (100). In the example of FIG. 10, the outer housing assembly (822) is equipped with a rib (824), which engages an activation lever (114) when the container and wicking assemblies (100) are inserted into the outer housing assembly (822). Once the rib (824) engages the activation lever (114), the rotation of the container and wicking assemblies (100) causes the rotational element (112) to rotate relative to the container assembly, which may break an internal barrier (106), allowing the volatile fluid (104) contained within the reservoir (102) to access the wick (110) of the wicking assembly. The volatile fluid (104) may then saturate the wick (110) by capillary action, and may then evaporate into the surrounding environment through ventilation holes (1028) in the outer housing assembly (822).

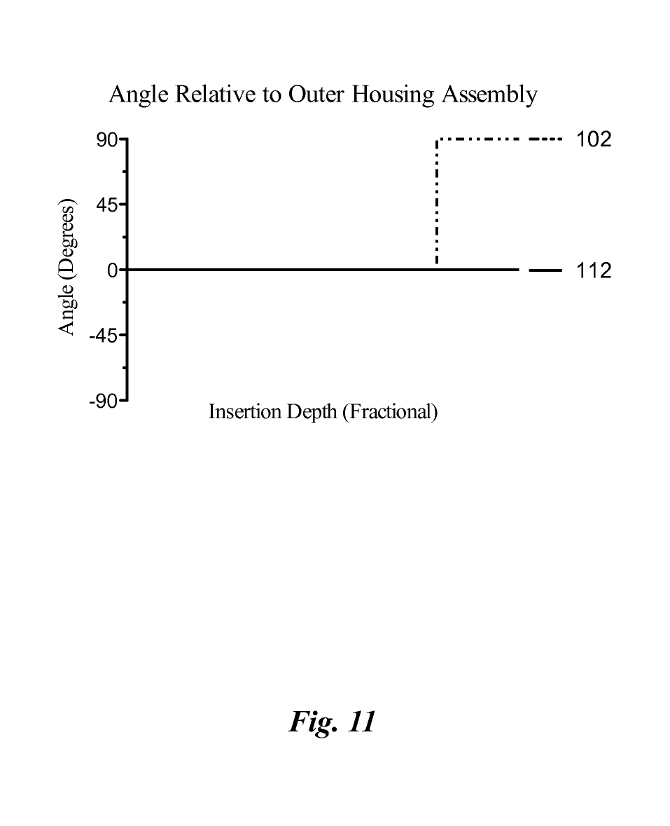

FIGS. 11-14 are charts showing the orientation of the reservoir (FIG. 1, 102) of the container assembly and the rotational element (FIG. 1, 112), both shown relative to the outer housing assembly (FIG. 8, 822), as a function of the insertion depth of the container and wicking assemblies (FIG. 1, 100) into the outer housing assembly (FIG. 8, 822). In FIGS. 11-13, the horizontal axis showing the insertion depth is not shown.

FIG. 11 is a chart showing the angle of a reservoir (FIG. 1, 102) of a container assembly and a rotational element (FIG. 1, 112) as a function of insertion depth relative to an outer housing assembly (FIG. 8, 822), according to an example of the principles described herein. In the chart shown in FIG. 11, the container and wicking assemblies (FIG. 1, 100) are inserted into the outer housing assembly (FIG. 8, 822); the activation lever (FIG. 1, 114) of the rotational element (FIG. 1, 112) is engaged, and once the container assembly is sufficiently inserted into the outer housing assembly (FIG. 8, 822), the container assembly is rotated. Because the rotation of the rotational element (FIG. 1, 112) is coupled to the outer housing assembly (FIG. 8, 822), the rotational element (FIG. 1, 112) rotates within the container assembly, causing the activation of the volatile fluid dispenser (FIG. 1, 100). As can be seen in FIG. 11, the rotational element (FIG. 1, 112) maintains a constant angle relative to the outer housing assembly (FIG. 8, 822) at all insertion depths. The reservoir (FIG. 1, 102) of the container assembly rotates relative to the outer housing assembly (FIG. 8, 822) as the container assembly is rotated. As a result, the rotational element (FIG. 1, 112) is rotated relative to the container assembly, and the volatile fluid dispenser (FIG. 1, 100) may be activated.

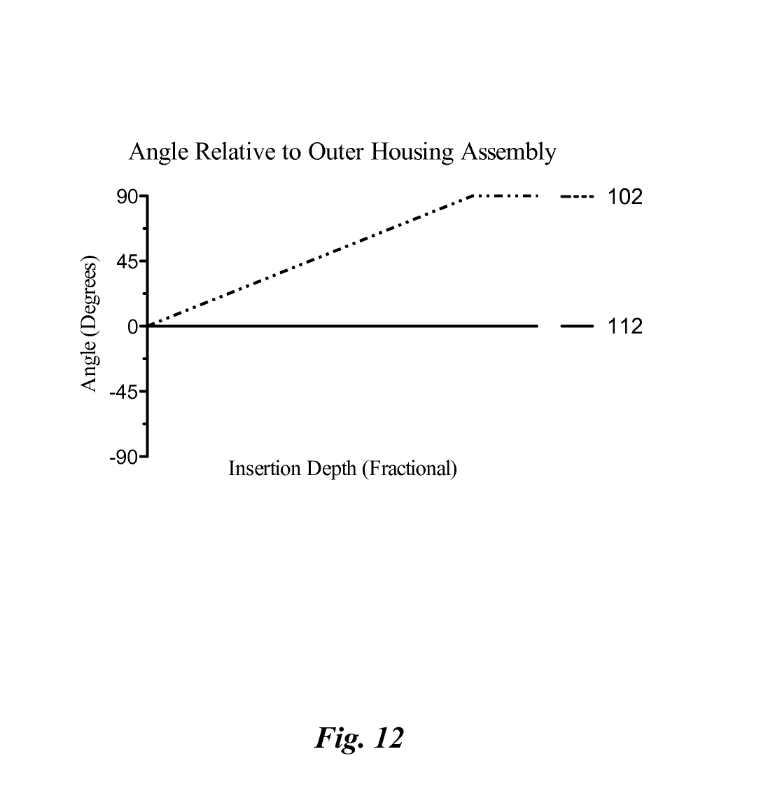

FIG. 12 is a chart showing the angle of a reservoir (FIG. 1, 102) of a container assembly and a rotational element (FIG. 1, 112) as a function of insertion depth relative to an outer housing assembly (FIG. 8, 822), according to an example of the principles described herein. The chart shown in FIG. 12 indicates a situation in which the outer housing assembly (FIG. 8, 822) engages the activation lever (FIG. 1, 114) of the rotational element (FIG. 1, 112), causing the rotational element (FIG. 1, 112) to be stationary (with respect to rotation, using the outer housing assembly (FIG. 8, 822) as the reference point). The chart shown in FIG. 12 may indicate the use of ribs that guide the container assembly to gradually rotate as the container and wicking assemblies (FIG. 1, 100) are inserted into the outer housing assembly (FIG. 8, 822). As a result, when the container assembly is fully inserted into the outer housing assembly (FIG. 8, 822), the rotational element (FIG. 1, 112) has rotated approximately 90° relative to the container assembly (as a consequence of the rotation of the container assembly, using the outer housing assembly (FIG. 8, 822) as the reference for said rotation). As a consequence of this rotation, when the container assembly is fully inserted into the outer housing assembly, the volatile fluid dispenser (FIG. 1, 100) may be activated.

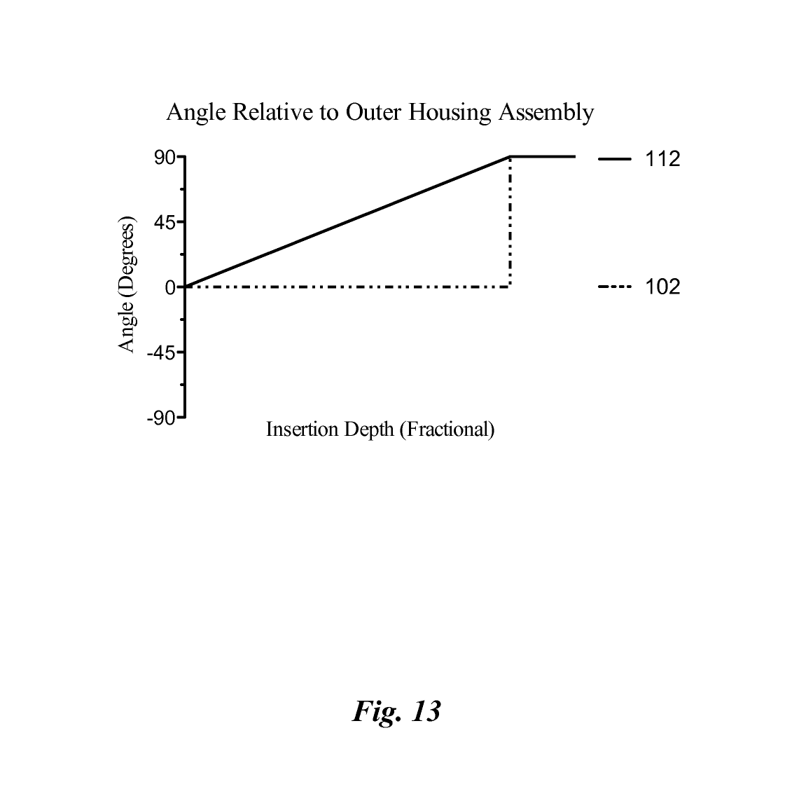

FIG. 13 is a chart showing the angle of a reservoir (FIG. 1, 102) of a container assembly and a rotational element (FIG. 1, 112) as a function of insertion depth relative to an outer housing assembly (FIG. 8, 822), according to an example of the principles described herein. The chart shown in FIG. 13 may indicate a situation in which a rib (FIG. 8, 824) engages the activation lever (FIG. 1, 114) of the rotational element (FIG. 1. 112), and the rib (FIG. 8, 824) guides the rotation of the rotational element (FIG. 1, 112) as the container and wicking assemblies (FIG. 1, 100) are inserted into the outer housing assembly (FIG. 8, 822). The rotation of the rotational element (FIG. 1, 112) relative to the reservoir (FIG. 1, 102) of the container assembly (whose relative rotation may be shown in degrees in FIG. 13 as the vertical distance between the solid trace (112) and the dashed trace (102)) causes a number of activation tabs (FIG. 1, 116) on the rotational element (FIG. 1, 112) to break an internal barrier (FIG. 1, 106) separating a wick (FIG. 1, 110) from a volatile fluid (FIG. 1, 104) contained within a reservoir (FIG. 1, 102). Once the container assembly is fully inserted into the outer housing assembly (FIG. 8, 822), which causes the activation of the volatile fluid dispenser (FIG. 1, 100), the container assembly may be similarly rotated 90°. The rotation of the container assembly causes the rotational element (FIG. 1, 112) to be re-aligned to its original position relative to the reservoir (FIG. 1, 102) of the container assembly (its pre-activation orientation). However, since the rotational element (FIG. 1, 112) was caused to rotate relative to the reservoir (FIG. 1, 102) of the container assembly as a consequence of the insertion of the container and wicking assemblies (FIG. 1, 100) into the outer housing assembly (FIG. 8, 822), the internal barrier (FIG. 1, 106) separating the wick (FIG. 1, 110) from the volatile fluid (FIG. 1, 104) within the reservoir (FIG. 1, 102) has been broken, and the volatile fluid dispenser (FIG. 1, 100) is activated. This example shows that the breakage of the internal barrier (FIG. 1, 106) separating the wick (FIG. 1, 110) from the volatile fluid (FIG. 1, 104) within the reservoir (FIG. 1, 102) may be irreversible, and also shows that the rotational element (FIG. 1, 112) may continue to rotate, or may rotate back to its original position (relative to the container assembly) following activation of the volatile fluid dispenser (FIG. 1, 100).

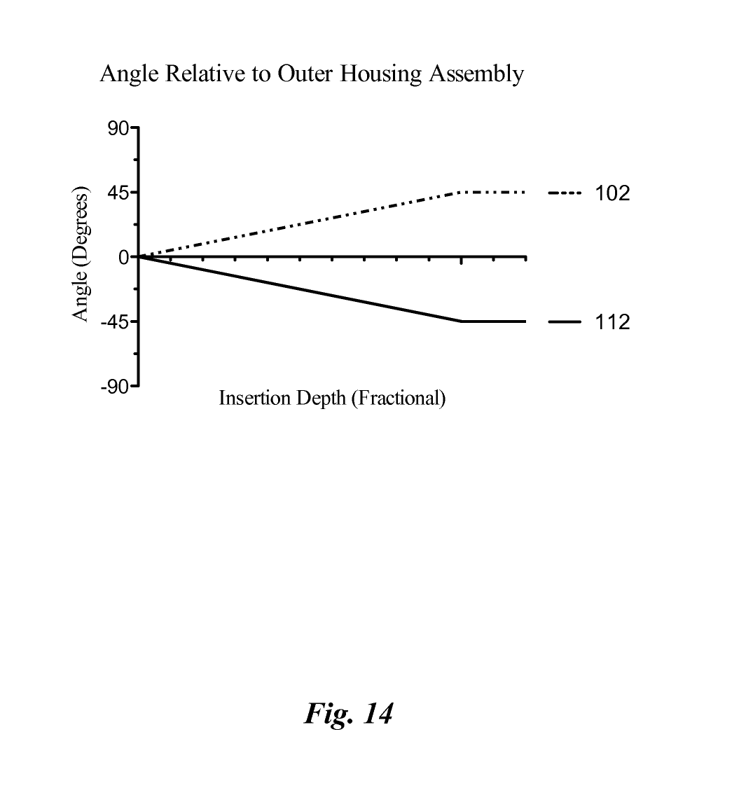

FIG. 14 is a chart showing the angle of a reservoir (FIG. 1, 102) of a container assembly and a rotational element (FIG. 1, 112) as a function of insertion depth relative to an outer housing assembly (FIG. 8, 822), according to an example of the principles described herein. FIG. 14 shows an insertion-depth axis. The chart shown in FIG. 14 may indicate a situation in which a rib (FIG. 8, 824) engages the activation lever (FIG. 1, 114) of a rotational element (FIG. 1, 112), and another rib may engage an element of the container assembly. These ribs guide the rotation of the reservoir (FIG. 1, 102) of the container assembly and the rotational element (FIG. 1, 112) in opposite directions during the insertion of the container and wicking assemblies (FIG. 1, 100) into the outer housing assembly (FIG. 8, 822). The reservoir (FIG. 1, 102) of the container assembly moves through an angle of approximately 45° (relative to the outer housing assembly (FIG. 8, 822)) as a consequence of the insertion of the container assembly into the outer housing assembly (FIG. 8, 822). Similarly, the rotational element (FIG. 1, 112) moves through an angle of approximately 45° (relative to the outer housing assembly (FIG. 8, 822), in the opposite direction from the rotation of the reservoir (FIG. 1, 102) of the container assembly) as a consequence of the insertion of the container and wicking assemblies (FIG. 1, 100) into the outer housing assembly (FIG. 8, 822). The result of the rotation of the container assembly and the rotational element (FIG. 1, 112) (which, in this example, are of equal magnitude but in opposite directions) causes the rotational element (FIG. 1, 112) to move through an angle of approximately 90° relative to the container assembly. Thus, as with the previous examples, activation tabs (FIG. 1, 116) on the rotational element (FIG. 1, 112) may break an internal barrier (FIG. 1, 106), allowing access of the volatile fluid (FIG. 1, 104) to the wick (FIG. 1, 110) and activating the volatile fluid dispenser (FIG. 1, 100).

FIGS. 11-14 diagram a number of scenarios in which the insertion of the container and rotational elements into the outer housing assembly may be used to activate the volatile fluid dispenser (FIG. 1, 100). The scenarios diagramed by FIGS. 11-14 are exemplary in nature, and do not represent every type of activation means which may be used according to the present specification. In some examples, the rotational element (FIG. 1, 112) may not rotate through an angle of 90°. In one example, the rotational element (FIG. 1, 112) may rotate through an angle of approximately 60°, and may similarly activate the volatile fluid dispenser (FIG. 1, 100) through said rotation. In another example, the rotational element (FIG. 1, 112) may rotate through an angle of 120° or 180°, and may activate the volatile fluid dispenser (FIG. 1, 100) through that rotation.

In another example of a volatile fluid dispenser (FIG. 3, 100) according to the present specification, the outer housing assembly may provide a base on which the container and wicking assemblies rest. In this example, the wick (FIG. 3, 110) of the wicking assembly may be protected by a cap during storage, but is not enclosed by any portion of the outer housing assembly upon activation. Such a cap may prevent the accumulation of dust, but the premature release of the volatile fluid is prevented by the activation assembly, rather than by the cap. The base provided by the outer housing assembly may be removable, or may be connected to the container or wicking assembly. As with the previous examples, the rotation of the container assembly relative to the outer housing assembly may cause the rotation of a rotational element (FIG. 3, 112) relative to the container assembly, whereby activating the volatile fluid dispenser (FIG. 3, 100). The engagement of the outer housing assembly and the rotational element (FIG. 3, 112) may be direct (e.g. by tabs or teeth protruding from the base of the container or wicking assembly which are connected to the rotational element (FIG. 3, 112)), or may be indirect (e.g. by tabs or teeth which are linked through a gearing mechanism). In either case, the tabs that engage the base (the outer housing assembly) with the rotational element (FIG. 3, 112) may be considered an activation lever. Accordingly, the rotation of the outer housing assembly (relative to the container assembly) may cause either a corresponding rotation of the rotational element (FIG. 3, 112), or may be increased or decreased by a constant factor due to a gearing mechanism. For example, the rotation of the outer housing assembly relative to the container assembly through an angle of 360° may cause the rotational element (FIG. 3, 112) to rotate 90° relative to the container assembly.

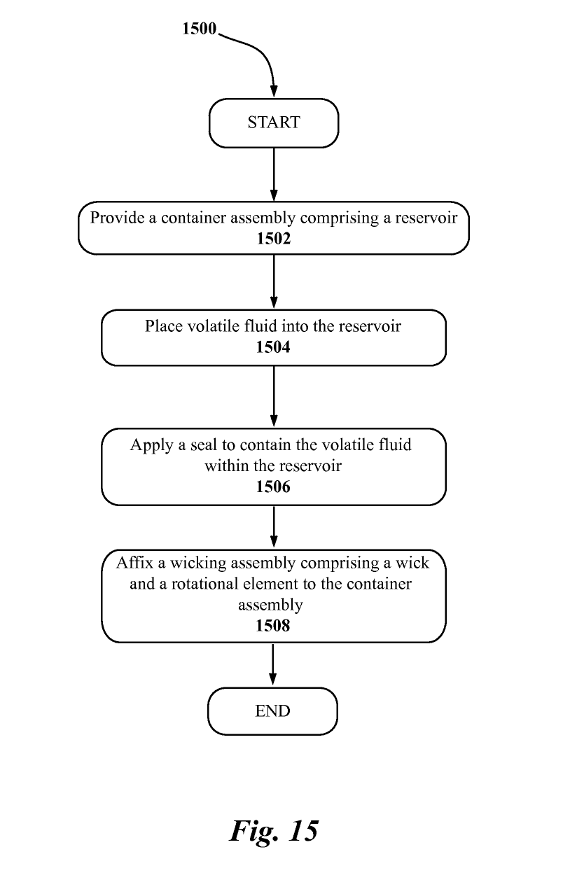

The present specification also provides a method to produce a device according to the present specification, which may be activated by a rotational force. FIG. 15 is a flowchart of a method (1500) of making a device according to an example of the principles described herein. Such a device may include a container assembly and a wicking assembly, and may also include an outer housing assembly.

The method (1500) involves providing (block 1502) a container assembly that includes a reservoir, placing (block 1504) a fluid into the reservoir, which reservoir may be part of an assembly. The method (1500) also involves applying (block 1506) a seal to contain the fluid within the reservoir. The method (1500) further involves affixing (block 1508) a wicking assembly that includes a wick and a rotational element to the container assembly. The assembly produced by operations 1502-1508 is such that the exertion of a rotational force on the rotational element causes the rotational element to rotate. The rotation of the rotational element causes an internal barrier separating the wick and the fluid contained within the reservoir to break, whereby activating the device. The assembly produced by operations 1502-1508 may be a combined container and wicking assembly.

Additionally, the method (1500) may further comprise additional operations. Such additional operations may include affixing a mechanism to rotate the rotational element. Such a mechanism to rotate the rotational element may provide an outer housing assembly. The outer housing assembly may be disposed beneath, above, or around the container assembly (or combinations thereof).

The operations of the method (1500) may be performed in any order. For example, assembling the container and wicking assemblies shown in FIGS. 3-5 may involve affixing the wicking assembly to the container assembly prior to filling the reservoir with a fluid. Conversely, assembling the container and wicking assemblies shown in FIGS. 1-2 may involve filling the reservoir with a fluid prior to affixing the wicking assembly. Further, it is also possible to affix the wicking assembly piecemeal, such as by affixing a rotational element to the container assembly, filling the reservoir with a fluid, sealing the reservoir to contain the fluid, and then affixing a wick to the combined container and wicking assembly.

The present specification also provides for a consumer product for dispensing a volatile fluid. The consumer product may include a container assembly, a wicking assembly and an outer housing assembly. The container assembly may include a reservoir that is sealed to contain a volatile fluid, and the sealed reservoir may include an internal barrier. The wicking assembly may include a wick, a rotational element, and may also include a fitment. The outer housing assembly may include a rib that engages the rotational element of the wicking assembly, and may also include a slot into which the container assembly and the wicking assembly may be inserted. The container assembly, wicking assembly, and outer housing assembly may be provided such that the engagement of the rib on the outer housing assembly with the rotational element of the wicking assembly couples the rotation of the rotational element to the rotation of the outer housing assembly, relative to the rotation of the container assembly. The rotation of the rotational element relative to the container assembly may break the internal barrier of the container assembly, and the breakage of the internal barrier of the container assembly may allow the volatile fluid contained in the container assembly to contact the wick of the wicking assembly.

The consumer product for dispensing a volatile fluid may also be provided with a number of activation levers, which may be part of the rotational element of the wicking assembly. The rotational element of the wicking assembly may also include a number of activation tabs. The outer housing assembly of the consumer product may also include a number of ventilation openings, which may allow the volatile fluid to evaporate into the surrounding environment.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A device for activating a volatile reservoir, comprising:
a container assembly, comprising:
a reservoir that is sealed to prevent an escape of a volatile fluid; and
an internal barrier; and
a wicking assembly, comprising:
a wick; and
a rotational element;
in which rotation of the rotational element:
breaks the internal barrier of the container assembly; and
allows the volatile fluid in the reservoir to contact the wick.

2. The device of claim 1, in which the volatile fluid in the container assembly comprises a fragrance, an aromatherapeutic agent, an insecticide, or a repellant.

3. The device of claim 1, in which the internal barrier is flexible.

4. The device of claim 1, in which the internal barrier is rigid.

5. The device of claim 1, in which the wick is a porous solid.

6. The device of claim 1, in which the wicking assembly further comprises a wicking fitment which guides the rotation of the rotational element.

7. The device of claim 1, in which the internal barrier comprises a reservoir fitment which guides the rotation of the rotational element.

8. The device of claim 1, in which the rotational element comprises an activation lever.

9. The device of claim 1, in which the rotational element comprises an activation tab which breaks the internal barrier upon rotation of the rotational element.

10. The device of claim 1, further comprising an outer housing assembly to contain the reservoir and wicking assembly.

11. The device of claim 10, in which the joining of the outer housing assembly to the container assembly and wicking assembly breaks the internal barrier of the container assembly.

12. The device of claim 1, in which the wicking assembly further comprises an emanator pad.

13. A method to produce an apparatus to activate a reservoir, comprising:
providing a container assembly comprising a reservoir;
placing a fluid into the reservoir;
applying a seal to contain the fluid within the reservoir; and
affixing a wicking assembly comprising a wick and a rotational element to the container assembly;
in which:
the rotation of the rotational element breaks an internal barrier separating the fluid in the reservoir from the wick of the wicking assembly.

14. The method of claim 13, further comprising applying a second seal to prevent leakage of the fluid following breakage of the internal barrier.

15. The method of claim 14, in which the second seal remains intact upon rotation of the rotational element.

16. The method of claim 13, in which the internal barrier that is broken upon rotation of the rotational element is included with the seal that is applied to contain the fluid within the reservoir.

17. The method of claim 13, in which the wicking assembly is affixed to the container assembly before placing a fluid into the reservoir.

18. A consumer product for dispensing a volatile fluid, comprising:
a container assembly comprising a reservoir that is sealed to contain a volatile fluid, in which the sealed reservoir comprises an internal barrier;
a wicking assembly comprising a wick, a rotational element, and a fitment; and
an outer housing assembly comprising a rib to engage the rotational element and a slot into which the container assembly and the wicking assembly may be inserted;
in which:
the engagement of the rib with the rotational element couples the rotation of the rotational element to the outer housing assembly;
the rotation of the rotational element relative to the container assembly breaks the internal barrier of the container assembly; and
the breakage of the internal barrier of the container assembly allows the volatile fluid contained in the reservoir to contact the wick of the wicking assembly.

19. The consumer product of claim 18, in which the engagement of the rib with the rotational element occurs by an activation lever on the rotational element.

20. The consumer product of claim 18, in which the outer housing assembly comprises a number of ventilation openings.
